(19) United States
(12) Patent Application Publication
(10) Pub. No.: US 2007/0202158 A1
(43) Pub. Date: Aug. 30, 2007

(54) Methods Of Administering Water-Soluble Prodrugs Of Propofol For Extended Sedation

(75) Inventors: Barbara S. Slusher, Kingsville, MD (US); Krystyna Wozniak, Bel Air, MD (US)

Correspondence Address: BANNER & WITCOFF, LTD., 1100 13th STREET, N.W., SUITE 1200, WASHINGTON, DC, 20005-4051, US

(73) Assignee: MGI GP, Inc., Wilmington, DE (US)

(21) Appl. No.: 10/580,405

(22) PCT Filed: Dec. 17, 2004
(86) PCT No.: PCT/US04/42301

§ 371 Date: Apr. 19, 2007

Related U.S. Application Data
(60) Provisional application No. 60/530,149, filed on Dec. 17, 2003.
(60) Provisional application No. 60/553,991, filed on Mar. 17, 2004.
(60) Provisional application No. 60/622,954, filed on Oct. 28, 2004.

(51) Int. Cl. A61K 9/48; A61K 9/24; A61K 31/66

(52) U.S. Cl. 424/451; 424/472; 514/130

(57) ABSTRACT

A method of administering a prodrug of propofol, preferably O-phosphonooxymethyl propofol disodium salt, comprises the oral, intragastric, or intraintestinal administration of the prodrug in amounts sufficient to induce or maintain a generalized anesthetized state, a conscious sedated state, or to treat insomnia, anxiety, nausea, vomiting, pruritus, epilepsy, and a range of pain syndromes and other medical conditions.

### FIELD OF THE INVENTION

The invention relates to methods of administering prodrugs of propofol (2,6-diisopropylphenol), a low molecular weight phenol derivative that is widely used as a hypnotic or sedative agent for intravenous administration in the induction and maintenance of anesthesia or sedation in humans and animals. Among its useful characteristics as an anesthetic drug are: administration via the intravenous route, rapid onset and offset of anesthesia, rapid clearance, and a side-effect profile that makes it preferable to other injectable anesthetics, such as barbiturates.

### BACKGROUND OF THE INVENTION

The use of injectable anesthetic agents generally, and of propofol specifically, in the induction and maintenance of general anesthesia has gained widespread acceptance in anesthetic care over the last 15 years. Intravenous anesthesia with propofol has been described to have several advantages over preexisting methods, such as more readily tolerated induction, since patients need have no fear of masks, suffocation, or the overpowering smell of volatile anesthetics; rapid and predictable recovery; readily adjustable depth of anesthesia by adjusting the IV dose of propofol; a lower incidence of adverse reactions as compared to inhalation anesthetics; and decreased dysphoria, nausea, and vomiting upon recovery from anesthesia [Padfield N L, Introduction, history and development. In: Padfield N L (Ed.) Ed., Total Intravenous Anesthesia. Butterworth Heinemann, Oxford 2000].

In addition to its sedative and anesthetic effects, propofol has a range of other biological and medical applications. For example, it has been reported to be an anti-emetic [McCollum J S C et al., Anesthesia 43 (1988) 239], an anti-epileptic [Chilvers C R, Laurie P S, Anesthesia 45 (1990) 995], and an anti-pruritic [Borgeat et al., Anesthesiology 76 (1992) 510]. Anti-emetic and anti-pruritic effects are typically observed at subhypnotic doses, i.e. at doses that achieve propofol plasma concentrations lower than those required for sedation or anesthesia. Antiepileptic activity, on the other hand, is observed over a wider range of plasma concentrations [Borgeat et al., Anesthesiology 80 (1994) 642]. Short-term intravenous administration of subanesthetic doses of propofol has also been reported to be remarkably effective in the treatment of intractable migraine and nonmigrainous headache [Krusz J C, et al., Headache, 40 (2000) 224-230]. It has further been speculated that propofol may be useful as an anxiolytic [Kurt et al., Pol. J. Pharmacol. 55 (2003) 973-7], neuroprotectant {Velly et al., Anesthesiology 99 (2003) 368-75], muscle relaxant [O'Shea et al., J. Neurosci. 24 (2004) 2322-7] and, due to its antioxidant properties in biological systems, may further be useful in the treatment of inflammatory conditions, especially inflammatory conditions with a respiratory component, and in the treatment of neuronal damage related to neurodegeneration or trauma. Such conditions are believed to be associated with the generation of reactive oxygen species and therefore amenable to treatment with antioxidants [see, e.g. U.S. Pat. No. 6,254,853 to Hendler et al.]

Propofol typically is formulated for clinical use as a oil-in-water emulsion. The formulation has a limited shelf-life and has been shown to be sensitive to bacterial or fungal contamination, which has led to instances of postsurgical infections [Bennett S N et al., N Engl J Med 333 (1995) 147]. Due to the dense, white color of the formulation, bacterial or fungal contamination cannot be detected by visual inspection of the vial in the first instance.

Not only is propofol poorly water soluble, but it also causes pain at the injection site, which must often be alleviated by using a local anesthetic [Dolin S J, Drugs and pharmacology. In: N. Padfield, Ed., Total Intravenous Anaesthesia. Butterworth Heinemann, Oxford 2000]. Due to its formulation in a lipid emulsion, its intravenous administration is also associated with undesirable hypertriglyceridemia in patients, especially in patients receiving prolonged infusions [Fulton B and Sorkin E M, Drugs 50 (1995) 636]. Its formulation as a lipid emulsion further makes it difficult to co-administer other IV drugs. Any physical changes to the formulation, such as a change in lipid droplet size, can lead to changes in the pharmacological properties of the drug and cause side effects, such as lung embolisms.

It has further been reported that the use of propofol in anesthesia induction is associated with a significant incidence of apnea, which appears to be dependent on dose, rate of injection, and premedication [Reves, J G, Glass, P S A, Lubarsky D A, Nonbarbiturate intravenous anesthetics. In: R. D. Miller et al., Eds, Anesthesia. 5^{th} Ed. Churchill Livingstone, Pa., 2000]. Respiratory consequences of administering anesthetic induction doses of propofol, including a reduction in tidal volume and apnea, occur in up to 83% of patients [Bryson et al., Drugs 50 (1995) at 520]. Induction doses of propofol are also known to have a marked hypotensive effect, which is dose- and plasma concentration-dependent [Reves et al., supra]. The hypotension associated with peak plasma levels after rapid bolus injection of propofol sometimes requires the use of controlled infusion pumps or the breaking-up of the induction bolus dose into several smaller incremental doses. Further, the short duration of unconsciousness caused by bolus induction doses renders propofol suitable for only brief medical procedures. For all the above reasons, propofol for induction and/or maintenance of anesthesia must normally be administered in an in-patient setting under the supervision of an anesthesiologist, and is often considered inappropriate for use by non-anesthesiologists in an ambulatory or day case setting.

In addition to its use in induction and maintenance of anesthesia, propofol has been used successfully as a sedative to accompany either local or regional anesthesia in conscious patients. Its sedative properties have also been exploited in diagnostic procedures that have an unsettling effect on conscious patients, such as colonoscopy or imaging procedures. Propofol has also been used as a sedative in children undergoing diagnostic imaging procedures or radiotherapy. A recent development is that of patient-controlled sedation with propofol. This technique is preferred by patients and is as effective as anesthesiologist-administered sedation.

Compared with the widely used sedative midazolam or other such agents, propofol provided similar or better sedative effects when the quality of sedation and/or the amount of time that patients were at adequate levels of sedation were measured [see Fulton B and Sorkin E M, Drugs 50 (1995) 636]. The faster recovery and similar or less amnesia associated with propofol makes it an attractive alternative to other sedatives, particularly for patients requiring only short sedation. However, because of the potential for hyperlipidemia associated with the current propofol formulation, and the development of tolerance to its sedative effects, the usefulness of propofol for patients requiring longer sedation is less well established.

Due to its very low oral bioavailability, propofol in its commercially available formulations is generally recognized as not suitable for other than parenteral administration, and must generally be injected or infused intravenously. While propofol is administered intravenously in a clinical setting, it has been suggested that it could be delivered for certain indications via other non-oral routes, such as via inhalation using a nebulizer, transmucosally through the epithelia of the upper alimentary tract, or rectally in the form of a suppository [see, e.g. Cozanitis, D. A., et al., Acta Anaesthesiol. Scand. 35 (1991) 575-7; see also U.S. Pat. Nos. 5,496,537 and 5,288,597]. However, the poor bioavailability of propofol when administered by any other than the intravenous route has hampered the development of such treatments. Alternative, safe, and simple methods of administration of propofol which do not require intravenous injections or infusions would be highly useful in a non-clinical setting for the treatment of conditions such as, for example, migraine and other severe headaches, trigeminal facial or dental pain, or arachnoiditis, to achieve mild sedation, anxiolysis, suppression of nausea, or as a sleep aid in individuals in need thereof. International patent application publication WO 02/13810 to Hendler teaches several propofol or di-propofol phosphate esters and carboxylic hemiesters of propofol which are disclosed as water-soluble and useful in the treatment of migraine.

Methods allowing for particularly the oral administration of propofol would be highly beneficial; to date, however, these medical needs have gone unmet. For all the reasons given above, there exists a clear clinical need for stable formulations of safe, orally bioavailable agents in anesthetic care, and for the treatment of conditions such as epilepsy, pruritus, and migraine and other severe headaches.

The development of water soluble and stable prodrugs of propofol, which is described in U.S. Pat. No. 6,204,257 to Stella et al., has made it possible to address these heretofore unmet needs, and to explore the pharmaceutical advantages of an orally bioavailable aqueous propofol-prodrug as a therapeutic agent. The prodrugs of the present invention differ from propofol in that the 1-hydroxy-group of propofol is replaced with a phosphonooxymethyl ether group: 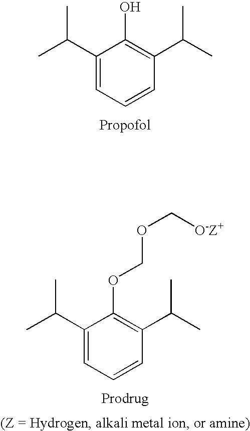
While the present invention is not bound by any theory, the prodrug is believed to undergo hydrolysis by endothelial cell surface alkaline phosphatases to release propofol.

Stella reports that the prodrug has good stability at pH levels suitable for making pharmaceutical formulations, and quickly breaks down in vivo under physiological conditions when administered intravenously. Unexpectedly, the inventors have now found that the prodrug can be administered orally to achieve a condition ranging from mild sedation and reduced responsiveness to external stimuli to deep sedation and loss of consciousness, depending on the orally administered dose of the prodrug. Another novel finding of the inventors is that the prodrug causes a rapid onset of the sedated/unconscious state after ingestion, followed by a plateau effect which is reached within 5-20 minutes after ingestion and is, depending on the dose and route of administration, maintained for up to one to four hours or longer. Yet another finding of the inventors is that the prodrugs of the invention display a high biological potency when administered directly into the duodenal lumen. When the upper alimentary tract—the oral cavity, pharynx, and stomach—is “bypassed,” the prodrug can be given in markedly lower doses than would be required to achieve a substantially similar pharmacological effect with intragastric administration. The prodrugs of the invention thus possess excellent and unexpected properties for oral administration and a favorable pharmacological profile as orally bioavailable therapeutics for sedation and anesthetic care, and for the treatment of conditions such as migraine, epilepsy, pruritus, anxiety, insomnia, nausea, and other medical conditions.

### SUMMARY OF THE INVENTION

The present invention provides a method of administering a compound to a patient in need thereof, comprising: orally or intragastrally administering a compound of Formula I in an amount sufficient to cause a pharmacological effect in said patient: 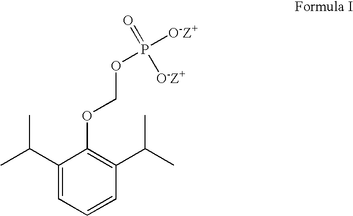
wherein each Z is independently selected from the group consisting of hydrogen, alkali metal ion, and amine. As stated above, the compound is capable of causing a pharmacological effect in a patient when administered intravenously, and a substantially similar pharmacological effect when administered orally or intragastrally in a higher dose. Thus, in this method of the invention, the orally or intragastrally administered amount is higher than the amount that would be sufficient to cause a substantially similar pharmacological effect by intravenous administration.

In a preferred aspect of this method of the invention, each Z in said compound of Formula I is an alkali metal ion. Preferably, the compound of Formula I is administered orally, and is formulated in a solid oral pharmaceutical formulation. Optionally, the solid oral pharmaceutical formulation is adapted to release a sufficient amount of the compound directly into the stomach after ingestion. Alternative formulations, useful for example if the compound is to be administered intragastrally through a nasogastric tube or other suitable catheter, include liquid formulations comprising the compound-of-formula I in-an-aqueous dissolved form, or in a slurry or suspension comprising granules or particles which in turn comprise the compound of formula I. These formulations can be further adapted to allow for specific desired release characteristics of the effective amount of the compound from the formulation directly into the stomach, such as fast release or sustained release over time.

An alternative method of administering a compound of Formula I to a patient in need thereof comprises introducing the compound directly into the gut. The compound is administered in an amount sufficient to cause a pharmacological effect in said patient. As stated above, the compound of formula I, when introduced directly into the gut, shows a biological potency that approaches that of, and is in the range of, potencies achievable also with intravenous administration. Thus, the administered dose in this alternative embodiment of the invention need not be higher than the intravenous dose sufficient to cause a substantially similar pharmacological effect. This means that the dose for administration directly into the gut to achieve a pharmacological effect is not defined relative to the intravenous dose sufficient to achieve a substantially similar pharmacological effect. “Introducing directly into the gut” means that the compound is administered to the patient in a way that “bypasses” the upper alimentary tract—the oral cavity, the pharynx, and the stomach—and that pharmacologically effective amounts of the compound of Formula I enter, or are released into, the digestive tract only at the level of the duodenum (the upper small intestine) or lower. The compound is introduced directly into the gut preferably by administering it orally in a specifically adapted pharmaceutical formulation. The formulation is specifically adapted to release a sufficient amount of the compound from the formulation only after it has passed through the upper alimentary tract. Preferred examples of such formulations are solid oral dosage forms such as enteric coated tablets, enteric coated capsules, or capsules or tablets comprising enteric coated granules or particles which in turn comprise the compound of Formula I, optionally adapted to allow for immediate or sustained release of the compound from the formulation. Alternative oral dosage forms for practicing this aspect of the invention are liquid, viscous, or semi-solid preparations comprising enteric coated granules or particles which in turn comprise the compound of Formula I. Alternatively, introduction of the compound directly into the gut is achieved by instilling a liquid preparation, preferably an aqueous solution, through a suitable catheter or tube.

The above described methods of administering the compound of Formula I to a patient, and any of the alternative or preferred embodiments thereof, include the administration of a dose sufficient to achieve a pharmacological effect in a patient. A range of doses can be selected depending largely on the pharmacological effect to be achieved. Preferred doses include those sufficient to induce or maintain an unconscious state; to induce or maintain a conscious sedated state; to induce or maintain a somnolent state, to treat insomnia, to treat sleep disorders characterized by inappropriate wakefulness; to treat anxiety; to treat nausea or vomiting; to treat itching associated with a pruritic condition; to treat an epileptic condition; to treat migraine pain; to treat cluster headaches, to treat other acute headaches, to treat trigeminal facial pain, to treat dental pain, to treat neuropathic pain, to treat phantom limb pain; to treat postoperative pain; to treat inflammatory pain; to treat neurogenic pain; and to treat arthritic pain.

One of the new and useful findings of the inventors is that the compounds of Formula I can be administered orally. One aspect of the invention is directed to administering a compound of Formula I employing a range of defined doses, without being limited to the specific purpose for which they are administered. Persons of ordinary skill in the art can determine, without undue experimentation, at which dose a compound of Formula I causes a pharmacological effect (including the specific pharmacological effects recited above), and thus select appropriate doses for use in the methods of this invention. For those embodiments of the invention which require that oral or intragastral doses be higher than intravenous doses, one skilled in the art can determine the intravenous dose sufficient to cause a pharmacological effect, and then introduce a higher dose of the compound into the stomach via the oral or intragastral routes to cause a substantially similar pharmacological effect. These steps require no more than routine experimentation by those skilled in the art, especially in light of the guidance and exemplary doses provided herein, and can all be done within the bounds of the invention.

In addition, the methods of this invention include methods for inducing or maintaining general anesthesia, for inducing or maintaining a conscious sedated state, and for treating a range of medical disorders such as the ones enumerated above. In a method for treating or preventing pain, a sufficient amount of the compound of Formula I is orally or intragastrally administered to a patient in need thereof. Preferred embodiments of this aspect of the invention include methods of treating or preventing migraine pain, cluster headaches, other acute headaches, trigeminal facial pain, dental pain, neuropathic pain, phantom limb pain; postoperative pain, inflammatory pain, neurogenic pain, and arthritic pain. Preferably, in these methods of treating pain syndromes, the compound of Formula I is administered orally in a pharmaceutical formulation that allows for the release of the compound directly into the gut, more preferably in the form suitable enteric coated dosage forms.

### BRIEF DESCRIPTION OF THE DRAWINGS

FIG. 1 illustrates the sedative/anesthetic effects of various doses of a compound of Formula I, O-phosphonooxymethyl propofol disodium salt, formulated as a 35 mg/ml w/v aqueous solution, on rats following oral/intragastric administration. Administration of the experimental compound caused a rapid onset (within 5-20 minutes of oral gavage) of sedated behavior, the extent and duration of which depended on the administered dose;

FIG. 2 illustrates the sedative/anesthetic effects of various doses of the experimental compound when formulated as a 200 mg/ml w/v aqueous solution, on rats following oral/intragastric administration. The experimental parameters were similar to those used to generate the results of FIG. 1;

FIG. 3 illustrates the sedative/anesthetic effects of various doses of the experimental compound when administered via the intravenous route; and

FIGS. 4a and 4b illustrate the sedative/anesthetic effects of the experimental compound when instilled directly into the gut via an intraduodenal catheter.

### DETAILED DESCRIPTION OF THE PREFERRED EMBODIMENTS

According to one embodiment of the present invention, an unconscious state is induced or maintained in a patient by the oral or intragastric administration of a prodrug of propofol in an amount sufficient to cause or maintain loss of consciousness. The prodrug is a compound of Formula I: 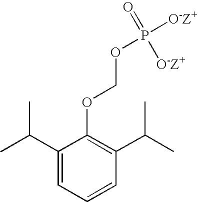
or a pharmaceutically acceptable salt thereof, wherein each Z is independently selected from the group consisting of hydrogen, alkali metal ion, and amine. Each Z preferably is an alkali metal ion, especially a sodium ion.

According to an alternative embodiment of the invention, the unconscious state is induced or maintained through administration of the compound of Formula I in a manner that bypasses the upper digestive tract and stomach, and introduces said compound directly into the intestinal tract. “Intestinal tract” means “gut,” i.e. that part of the alimentary tract which begins after the stomach and ends with the rectal cavity. This embodiment of the invention can be practiced, for example, by instilling a liquid preparation comprising the compound of Formula I into the gut, preferably the duodenum, by use of a suitable transgastric or transabdominal intraintestinal catheter. Alternatively, the compound of Formula I can be administered via the oral route in the form of suitable enteric coated preparations, such as, without limitation, enteric coated tablets; enteric coated capsules; or tablets, capsules, or liquid or semi-liquid preparations such as slurries or suspensions comprising enteric coated granules or other particles which in turn comprise the compound of Formula I. As will be appreciated by those skilled in the art, many techniques exist by which compounds such as those of Formula I may be formulated and/or administered in a way that prevents their release during passage through the mouth, esophagus, and stomach, and permits their introduction directly into the gut, and the examples recited herein by which such introduction may be achieved are not intended to be limiting in any way.

The compound of Formula I may be administered by itself or may be co-administered together with one or more additional active agents. Non-limiting examples of additional active agents include hypnotic, analgesic, anti-inflammatory, amnesic, muscle relaxant, and sedative agents. Such additional active agents may be incorporated into an orally or intragastrally administrable pharmaceutical composition comprising the compound of Formula I; or may be administered or formulated in a manner that allows their introduction directly into the gut together with a compound of Formula I; or may be administered in a separate pharmaceutical formulation.

Appropriate exemplary doses for inducing or maintaining an unconscious state in a patient by single or repeated oral or intragastric administration of the compound of Formula I range from about 100 mg/kg to about 1,000 mg/kg, preferably from about 200 mg/kg to about 600 mg/kg, and more preferably from about 250 mg/kg to about 500 mg/kg.

If the unconscious state is induced or maintained by administering the compound of Formula I in a manner that allows for its introduction directly in to the gut, for example by use of suitable enteric coated formulations or by intraduodenal instillation, suitable exemplary doses range from about 500 mg/kg to about 15 mg/kg, preferably from about 400 mg/kg to about 20 mg/kg, and more preferably from about 300 mg/kg to about 25 mg/kg.

As will be appreciated by those skilled in the art, many factors influence the choice of appropriate dosage, mode, and schedule of administration. For example, the appropriate dosage for inducing or maintaining an unconscious state in a patient, or for practicing any of the other methods of this invention recited below, may depend on whether the patient is a human, or another mammal, or is a non-mammalian patient; it may depend on the patient's age, weight, sex, diet, health, underlying medical condition, and the like. Therefore, an anesthesiologist, veterinarian, or other medical, science, or health practitioner skilled in the art will be able to devise, in light of the guidance provided herein, and without undue experimentation, an appropriate treatment protocol for practicing the present invention.

In another embodiment of the invention, a conscious sedated state is induced, or maintained over an extended period of time, in a patient by oral or intragastric administration of a compound of Formula I; or by administration of a compound of Formula I in a manner allows for its introduction directly into the gut, for example by use of suitable enteric coated formulations or by intraduodenal instillation.

In another embodiment of the present invention, a somnolent state is induced, or maintained over an extended period of time, in a patient. As is the case for a conscious sedated state, above, the somnolent state can be induced or maintained by orally or intragastrally administering an effective amount of a compound of Formula I; or by administering the compound in a manner that allows for its introduction directly into the gut, for example by use of suitable enteric coated formulations or by intraduodenal instillation.

Appropriate exemplary dose levels for inducing or maintaining a somnolent state in a patient by single or repeated oral or intragastral administration range from about 10 mg/kg to about 300 mg/kg, preferably from about 20 mg/kg to about 250 mg/kg, and more preferably from about 25 mg/kg to about 200 mg/kg. Dose levels sufficient to induce a conscious sedated state overlap with doses sufficient to induce a somnolent state, and range from about 20 mg/kg to about 400 mg/kg, preferably from about 20 mg/kg to about 300 mg/kg, more preferably from about 50 mg/kg to about 250 mg/kg, and yet more preferably from about 30 to about 70 mg/kg. For example, a patient in need of sedation may be administered a prodrug of Formula I, O-phosphonooxymethyl propofol disodium salt, orally in a pharmaceutical formulation that releases the prodrug into the stomach, at a dose ranging from more than 35 mg/ml to about 70 mg/ml.

If the somnolent state is induced or maintained by administering the compound of Formula I in a manner that allows for its introduction directly into the gut, for example by use of suitable enteric coated formulations or by intraduodenal instillation, suitable exemplary doses range from about 1 mg/kg to about 75 mg/kg, preferably about 2 mg/kg to about 50 mg/kg, and more preferably about 5 mg/kg to about 40 mg/kg. Dose levels sufficient to induce or maintain a conscious sedated state overlap with those required to induce or maintain a somnolent state, and range for example from about 2 mg/kg to about 100 mg/kg, preferably about 5 mg/kg to about 75 mg/kg, more preferably from about 10 mg/kg to about 50 mg/kg, yet more preferably from about 10 mg/kg to about 40 mg/kg, and even more preferably from about 15 to about 35 mg/kg.

The induction or maintenance of a somnolent state, experienced as e.g. a relaxed and mildly drowsy inclination to sleep, is desirable, for example, in individuals suffering from insomnia or another condition characterized by increased and inappropriate wakefulness relative to the demands of society, such as circadian rhythm sleep disorders (e.g. delayed sleep phase disorder, “jet lag”, or “shift work” type sleep disorder). For induction of a somnolent state, the compound of Formula I can be administered singly, or in combination with other sleep-inducing compounds, combined in a single oral formulation or separately.

Dose levels sufficient to induce a conscious sedated state or a somnolent state are further useful in the treatment of anxiety in patients in need of such treatment, as will be appreciated by those skilled in the art. Thus, anxiolytically effective doses of the compound of Formula I will be coextensive with doses which themselves cause conscious sedation or mild to moderate sleepiness, and can be administered to patients in need of anxiolytic therapy via the oral or intragastral routes; or in a manner that allows for the introduction of the compound directly into the gut, for example by use of suitable enteric coated formulations or intraduodenal instillation.

Those skilled in the art will appreciate that compounds of Formula I, while being useful in the induction and maintenance of anesthesia, sedation, sleep, and anxiolysis as described above, are also useful in treating other medical conditions known to be amenable to treatment with propofol. Therefore, there is provided in another aspect of this invention a method of suppressing nausea or vomiting in a patient, wherein a compound of Formula I is orally or intragastrally administered to a patient in an amount sufficient to suppress nausea or vomiting. Alternatively, the compound can be administered in a manner that allows for its introduction directly into the gut, for example by use of suitable enteric coated formulations or by intraduodenal instillation. This aspect of the invention has particular applications in settings where the patient suffers from, or is at risk of, nausea or vomiting related to cancer chemotherapy, or where the patient suffers from or is at risk for postoperative nausea and vomiting. Within this aspect of the invention, compounds of Formula I are preferably administered at subhypnotic doses, i.e. the dose of the compound of Formula I, whether administered orally, intragastrally, or in a manner that allows for its introduction directly into the gut, does not cause loss of consciousness, and, if the patient is not also in need of sedation, preferably does not cause a sedated state. For example, appropriate doses for suppressing or alleviating nausea and vomiting in a patient by single or repeated oral or intragastral administration range from about 2 mg/kg to about 250 mg/kg, preferably from about 5 mg/kg to about 200 mg/kg, more preferably from about 5 mg/kg to about 150 mg/kg, and yet more preferably from about 7.5 to about 30 mg/kg. For example, a patient suffering from nausea may be administered a prodrug of formula I, O-phosphonooxymethyl propofol disodium salt orally in a formulation that releases the prodrug directly into the stomach at a dose of more than 15 to about 30 mg/kg. Generally lower effective doses may be used if the compound is administered in a manner that allows for its introduction directly into the gut. Such exemplary doses range from about 1 mg/kg to about 50 mg/kg, preferably from about 2 mg/kg to about 30 mg/kg, more preferably from about 2 mg/kg to about 20 mg/kg, and even more preferably from about 3.5 to about 12.5 mg/kg.

Another aspect of the present invention provides a method of treating itching associated with a pruritic condition in a patient, wherein a compound of Formula I is orally or intragastrally administered to a patient in an amount sufficient to prevent, alleviate, or suppress localized or general itching. Alternatively, the compound may be administered in a manner that allows for its introduction directly into the gut, for example by intraduodenal instillation or by use of suitable enteric coated formulations. Within this aspect of the invention, compounds of Formula I are preferably administered at subhypnotic doses, i.e. the administered amount of the compound of Formula I does not cause loss of consciousness, and, if the patient is not also in need of sedation, preferably does not cause a sedated state. For example, appropriate doses for suppressing or alleviating local or generalized itching in a patient by single or repeated oral or intragastral administration range from about 2 mg/kg to about 250 mg/kg, preferably from about 5 mg/kg to about 200 mg/kg, more preferably from about 5 mg/kg to about 150 mg/kg, and even more preferably from about 7.5 mg/kg to about 30 mg/kg. For example, a patient suffering from generalized intractable itching may be administered a prodrug of formula I, O-phosphonooxymethyl propofol disodium salt, orally in a formulation that releases the prodrug directly into the stomach at a dose of more than 15 to about 30 mg/kg. If the compound is administered in a manner that allows for its introduction directly into the gut, lower effective doses may be used. Such exemplary doses range from about 1 mg/kg to about 50 mg/kg, preferably from about 2 mg/kg to about 30 mg/kg, more preferably from about 2 mg/kg to about 20 mg/kg, and even more preferably from about 3.5 mg/kg to about 12.5 mg/kg.

The compound of Formula I, or a pharmaceutically acceptable salt thereof, may be administered for treating patients suffering from an epileptic condition. A patient in need of such treatment is orally or intragastrally administered a dose of a compound of Formula I in an amount sufficient to prevent, suppress, or alleviate the epileptic condition. Alternatively, the compound may be administered in a manner that allows for its introduction directly into the gut, for example by intraduodenal instillation or by use of suitable enteric coated formulations. Suitable dosages for treating patients suffering from an epileptic condition range from subhypnotic doses, as defined above, to higher, hypnotic doses, as required by the individual patient's needs. Individual suitable doses can be determined by those skilled in the art, especially in light of the guidance provided herein. A suitable dose for an unconscious patient presenting with status epilepticus, for example, may be determined and adjusted as needed by monitoring brain seizure activity on an electroencephalogram, and a suitable liquid formulation comprising the compound of formula I may be administered via a nasogastric tube.

If an epileptic condition is to be treated by single or repeated oral or intragastric administrations of a compound of Formula I, for example, appropriate doses typically range from about 2 mg/kg to 400 mg/kg, more preferably from about 5 mg/kg to about 300 mg/kg, more preferably from about 5 mg/kg to about 200 mg/kg body weight, and even more preferably from about 7.5 mg/kg to about 60 mg/kg. If the epileptic condition is to be treated by administration of the compound of formula I in a manner that allows for its introduction directly into the gut, suitable exemplary doses range from about 1 mg/kg to about 100 mg/kg, preferably from about 1 mg/kg to about 75 mg/kg, more preferably from about 2 mg/kg to about 50 mg/kg, and even more preferably from about 3.5 to about 25 mg/kg body weight.

In another aspect, the present invention provides a method for treating migraine pain, cluster headaches, and other acute headaches. Patients in need of such treatment are orally or intragastrally administered an effective amount of a compound of Formula I, or of a pharmaceutically acceptable salt thereof, singly, or in repeated doses until pain relief is accomplished. Alternatively, the compound or its salt may be administered in a manner that allows for its introduction directly into the gut, such as by intraduodenal instillation, or by oral administration of suitable enteric coated formulations. Exemplary oral or intragastric doses suitable to practice this aspect of the invention range from about 2 mg/kg to about 300 mg/kg, preferably from about 5 mg/kg to about 250 mg/kg, and more preferably from about 5 mg/kg to about 200 mg/kg, and even more preferably from about 10 to about 30 mg/kg body weight. Such doses may be lowered when the compound is introduced directly into the gut, in which case typical exemplary doses range from about 1 mg/kg to about 75 mg/kg, preferably from about 1 mg/kg to about 50 mg/kg, more preferably from about 2 mg/kg to about 30 mg/kg, and even more preferably from about 5 mg/kg to about 20 mg/kg body weight. Since such doses overlap with antiemetic doses, above, they are also expected to be effective in treating nausea frequently associated with migraine pain.

As will be appreciated by those skilled in the art, pain syndromes other than acute headaches will also be treatable by oral or intragastric administration of the compounds of Formula I at the preferred dose levels described in the preceding paragraph, and the treatment of such other pain syndromes is intended to be within the scope of this invention. Non-limiting examples of such other pain syndromes are: trigeminal facial or dental pain; neuropathic pain associated with neuropathies caused by disease (e.g. diabetes, or viral infections such as herpes or HIV or drugs (e.g. taxol, cisplatin, and other anticancer agents); phantom limb pain suffered by amputees; persistent and largely intractable postoperative pain; and arthritic pain.

The present invention also provides a method for the treatment of a pathologic condition having an inflammatory component in a patient, wherein a pharmacologically effective amount of a compound of Formula I is orally or intragastrally administered to the patient. Alternatively, the compound may be administered in a manner that allows for its introduction directly into the gut, for example by intraduodenal instillation or by use of suitable enteric coated formulations. This embodiment of the invention finds particular application in the treatment of a pathologic condition of the nervous system having an inflammatory component.

In another aspect, the present invention provides a method for the treatment of a pathologic respiratory condition in a patient, wherein a pharmacologically effective amount of a compound of Formula I as defined above is orally or intragastrally administered to the patient. Alternatively, the compound may be administered in a manner that allows for its introduction directly into the gut, for example by intraduodenal instillation or by use of suitable enteric coated formulations. This embodiment of the invention finds particular application in pathologic respiratory conditions associated with oxidative tissue damage.

In another aspect, the present invention provides a method of treatment wherein a compound of Formula I as defined above is orally or intragastrally administered to a patient in conjunction with a cytostatic chemotherapeutic agent, and wherein the patient suffers from cancer. In this embodiment of the invention, the compound may alternatively be administered in a manner that allows for its introduction directly into the gut, for example by intraduodenal instillation or by use of suitable enteric coated formulations.

In another aspect, the present invention provides a method of treating spasticity, hyperekplexia, or of providing muscle relaxation in a patient in need thereof, which comprises orally administering to said patient a therapeutically effective amount of a compound of formula I, optionally in a pharmaceutical formulation that allows for the release of the effective amount of said compound directly into the gut. Suitable oral or intragastric doses to practice this aspect of the invention include the single or repeated oral administration of about 10 mg/kg to about 350 mg/kg, preferably from about 30 mg/kg to about 200 mg/kg, and more preferably from about 40 mg/kg to about 80 mg/kg body weight. If the compound is administered in a manner that allows for its introduction directly into the gut, for example by intraduodenal instillation or oral administration of enteric coated formulations, suitable doses range from about 5 mg/kg to about 200 mg/kg, preferably from about 20 mg/kg to about 125 mg/kg, and more preferably from about 30 mg/kg to about 50 mg/kg body weight.

In yet another aspect of the present invention, there is provided a method of preventing neurodegeneration in the central nervous system, which comprises: orally administering to a patient suffering from, or being at risk for, neurodegeneration caused by traumatic or vascular injury, toxicity, or disease, a therapeutically effective amount of a compound of formula I. Said therapeutically effective amount is optionally administered in a pharmaceutical formulation specifically adapted to release said compound directly into the stomach, or alternatively directly into the gut. As is the case for the other methods of treatment included in this invention, the formulation is optionally adapted to allow for immediate or fast release of the compound from the formulation, or for gradual, sustained release over time. In a preferred embodiment of this aspect of the invention, the patient suffers from, or is at risk of, ischemic injury to the brain, for example as a result of having suffered a stroke.

Skilled practitioners will appreciate that the methods of treating the various medical conditions described above comprise not only the administration of the prodrug of formula I to a patient who is already suffering the symptoms and effects of the condition, but also its administration to a patient who is at risk for developing or suffering from said conditions. For example, many migraineurs suffer from periodic or cyclic migraines that allow them to predict with reasonable accuracy certain times or periods during which they are likely to experience an attack, such as certain times during the menstrual, seasonal, or lunar cycle. Other migraineurs will point to specific triggering events, such as certain odors or stress. Many experience prodromal signs that tell them that the onset of a migraine attack is looming, or auras that signal that an attack is imminent. In treating such patients, the compound of formula I can be administered not only to relieve acute pain and shorten postdromal symptoms, but also to abort the onset of a migraine attack before pain onset, or even to prevent migraine symptoms from occurring altogether. In another example, it is well understood that a certain proportion of patients undergoing cancer chemotherapy or radiation therapy will suffer nausea and vomiting. The same holds true for patients who recover from general anesthesia, for migraineurs and other intractable headache sufferers, and for individuals who are prone to car- sea- or air-sickness. In such patients, known to be at recognized risk of suffering from nausea or vomiting, preventive treatment with antiemetic doses of the compound of formula I is expected to be efficient in suppressing the development of adverse symptoms. Thus, for all the medical conditions listed above, “treatment” includes not only the relief of acute symptoms, but also the prophylactic administration of suitable doses of the prodrug of formula I to patients who are not (yet) symptomatic, but who are at recognized risk.

Skilled practitioners will further appreciate that the specific doses described above can be administered at various intervals and regimens, as dictated by the individual patient's needs and by the nature of the condition to be treated. Thus, for inducing a conscious sedated state in a subject undergoing only a short surgical or diagnostic procedure, single or repeated administration of suitable bolus doses of the prodrug of formula I may be sufficient. In other patients, for example in cancer patients suffering from nausea during prolonged infusions of chemotherapeutic agents; in intensive-care burn patients requiring prolonged sedation; or in patients suffering from prolonged epileptic seizures, a sustained or continuous administration of the prodrug may be required. If the patient's needs so require, the above-exemplified doses should be understood as mg/kg/h in therapeutic settings where the prodrug is administered not in one or several discrete boli, but is instead delivered via continuous infusion through e.g. a suitable nasogastric or intraduodenal catheter. For example, a patient suffering from sustained epileptic seizures or status epilepticus may be treated with intragastric infusions ranging from about 2 to about 400 mg/kg/h or with intraduodenal infusions ranging from about 1 mg/kg to about 100 mg/kg/h; a patient suffering from sustained nausea or vomiting may be treated with intragastric infusions of doses ranging from about 2 to about 250 mg/kg/h, or with intraduodenal infusions of doses ranging from about 1 to about 50 mg/kg/h (or, in each case, with infusions of doses in any of the preferred doses ranges recited above).

Methods for the chemical synthesis of the propofol prodrug of Formula I from propofol are described in U.S. Pat. No. 6,204,257 to Stella et al., and are incorporated herein by reference in their entirety. The propofol prodrug of Formula I is water soluble and can be formulated in aqueous solutions or in other suitable pharmaceutical compositions.

As those in the art will appreciate, the compounds of Formula I can be readily formulated for oral administration by combining them with well-known pharmaceutically acceptable carriers. Such carriers enable the compounds of the invention to be formulated as tablets, pills, capsules, liquids, quick-dissolving preparations, gels, syrups, slurries, suspensions and the like, for oral ingestion by a patient to be treated. Pharmaceutical preparations for oral use can be obtained by mixing the compound with a solid excipient, optionally grinding a resulting mixture, and processing the mixture of granules, after adding suitable auxiliaries, if desired, to obtain tablets. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In general, the pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. If desired, disintegrating agents may be added, such as the cross-linked polyvinyl pyrrolidone, agar, or alginic acid or a salt thereof such as sodium alginate or a number of others disintegrants (see, for example, Remington's Pharmaceutical Sciences, Mack Publishing Co., Easton, Pa., Remington's Pharmaceutical Sciences, Mack Publishing, Easton, Pa., 20th Ed, 2000). For liquid formulations, any pharmaceutically acceptable aqueous medium may be used, such as sterile water, physiological saline, or a mixture of water and an organic solvent, such as propylene glycol, ethanol, and the like. The concentration of the compound of Formula I in the formulation most often ranges from about 0.5 to about 35% (w/v), more usually from about 1 to about 20%.

### EXAMPLE 1

This example compares the dose-dependent pharmacological effects of a propofol prodrug of Formula I, O-phosphonooxymethyl propofol disodium salt, on rats when administered in a single oral dose to the pharmacological effects observed after an equipotent intravenous infusion. Young adult male Sprague-Dawley rats (250-300 g, Charles River Laboratories) received oral doses of vehicle (0.12% Tris/0.25% monothioglycerol/saline; n=4, per oral gavage) or of O-phosphonooxy-methyl propofol disodium salt at doses of 100, 200, 300 and 400 mg/kg, dissolved in vehicle at 35 mg/ml (n=2 per dose) or 200 mg/ml w/v (n=2 per dose). The animals' behavior was then scored independently by two blinded but experienced observers in 5-minute intervals for 2 hours according to the following rating scale: 4=loss of consciousness; 3=moderate to deep sedation, markedly reduced responsiveness to external stimuli and slow but generally maintained postural reflexes; 2=“drowsy,” some slowing and sluggishness of postural reflexes but maintained responsiveness to external stimuli; 1=awake but passive, little to no locomotor or exploratory activity; 0=normal.

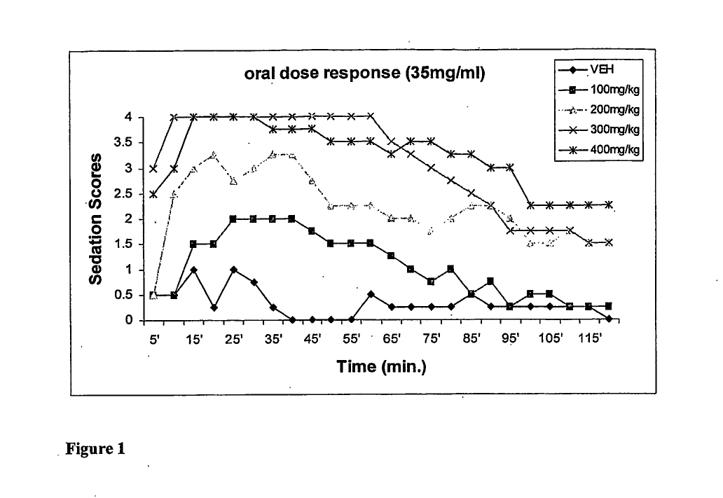

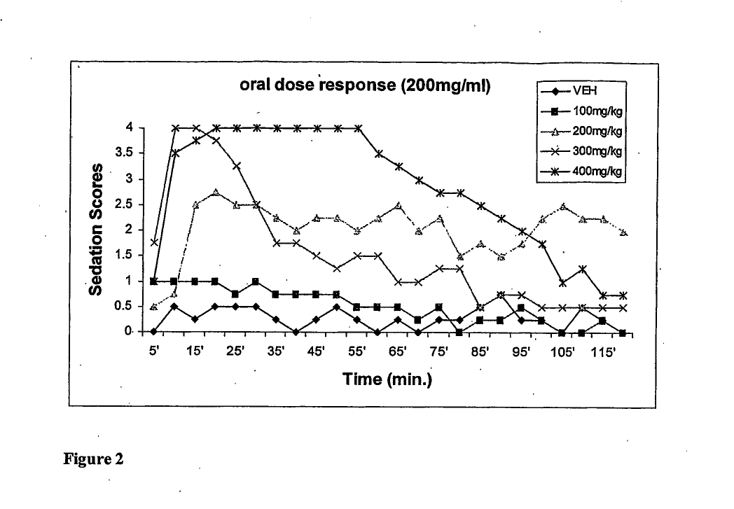

The results of this experiment are presented in FIGS. 1-3. Animals orally dosed with O-phosphonooxymethyl propofol disodium salt displayed a rapid (within 5-10 minutes of dosing) dose-dependent onset of sedated behavior, quickly followed by loss of consciousness in the 300 and 400 mg/kg dose groups (see FIGS. 1 and 2). Loss of consciousness lasted for up to about 1 hour in animals in the highest dose group. Compared to vehicle-dosed control animals, animals in the intermediate dose groups (100-200 mg/kg) displayed signs of mild to moderate sedation lasting for about 1-2 hours and longer following oral administration (see FIGS. 1 and 2). This study demonstrates that the tested prodrug of Formula I, O-phosphonooxymethyl propofol disodium salt, is orally bioavaliable and capable of causing a relatively long-lasting dose-dependent anesthetic or sedative effect with a rapid onset after ingestion.

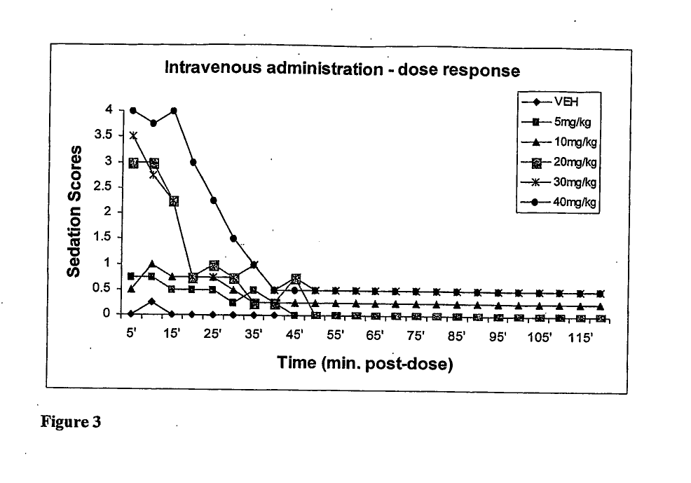

The above-described pharmacological effects of oral administration of the tested prodrug were compared with those caused by an equipotent intravenous infusion: Under halothane anesthesia, young adult rats received femoral vein catheters which were exteriorized and attached to a liquid swivel via a protectant spring. About 20 minutes after catheterization, and after full behavioral recovery from halothane anesthesia, each animal was attached to an electronic infusion pump and was administered vehicle, or 5, 10, 20, 30, or 40 mg/kg of the test prodrug (n=2 per dose) in 1 ml total volume by gradual constant-rate intravenous infusion over 10 minutes. Behavioral rating as described above began immediately following the end of infusion. The results of this experiment are illustrated in FIG. 3: As was the case for oral administration, above, intravenous administration of the prodrug of Formula I, O-phosphonooxymethyl propofol disodium salt, caused a rapid-onset dose-dependent sedated/anesthetized state, the depth and duration of which depended on the administered dose. Overall, the sedated/anesthetized state was maintained for shorter durations as compared to oral administration (see FIG. 3).

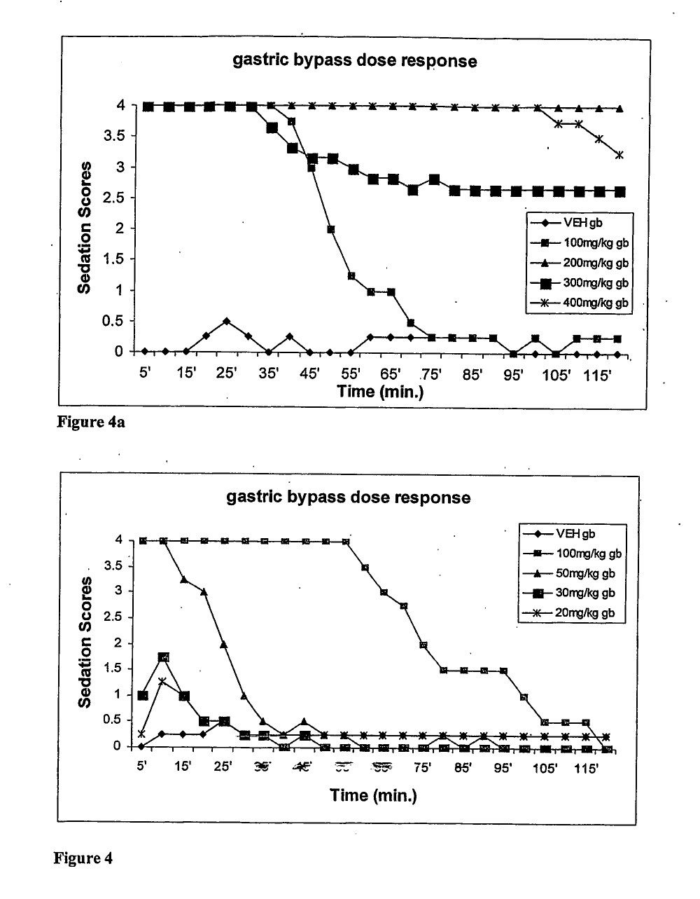

The above-described efficacy of the tested prodrug when administered orally or intravenously was compared to a mode of administration that allows for the introduction of the prodrug directly into the gut. For this “gastric bypass” experiment, young adult male Sprague Dawley rats (225-250 g body wt.) underwent implantation of intraduodenal catheters to allow for intraduodenal instillation of an aqueous buffered solution of the prodrug. Following full recovery from catheter implantation surgery, various concentrations of O-phosphonooxymethyl propofol disodium salt in vehicle, or vehicle alone, were administered via the catheter in a constant volume of 2 ml/kg body weight to yield doses of 0, 20, 30, 50, 100, 200, 300, and 400 mg/kg body weight (n=2 per dose). Behavioral assessment was conducted as described above. The results of this experiment are illustrated in FIG. 4: Within five minutes of intraduodenal administration, rats dosed with the prodrug but not vehicle displayed a rapid-onset sedative effect, quickly followed by loss of consciousness in the higher dose groups. The depth and duration of sedation, and the length of unconsciousness in the higher dose groups, depended on the administered dose.

Upon administration, the test prodrug is converted in the body into propofol, its pharmacologically active metabolite. The pharmacokinetic profile, i.e. the blood plasma concentration of propofol derived from the test prodrug, was assessed in a separate experiment. Male Sprague-Dawley Rats (225-250 g) were obtained with indwelling jugular or intraduodenal catheters (Hilltop Labs, PA). On the day of testing, control blood samples were taken from the jugular vein prior to dosing. O-phosphonooxymethyl propofol disodium salt was then dosed in different concentrations in groups of 2-3 rats. The test prodrug was administered either by the oral, intravenous or intraduodenal routes. Blood samples (0.5 ml) were taken at 5, 15, 30, 45, 60, 120, 240 and 360 min after administration of the test prodrug. Blood samples were centrifuged to obtain plasma and stored frozen until analysis. The outcome of this experiment is depicted in the following table I:

TABLE I
Bioavailability of propofol from O-Phosphonooxymethyl
propofol disodium salt for various methods of
administration, relative to 5 mg/kg IV

| | | | | AUCt | |
|---|---|---|---|---|---|
| | Dose | Cmax | F_Cmax | (μg × | F_AUCt |
| Route | mg/kg | (μg/mL) | % | min/mL) | % |
| I.V. | 5 | 0.286 | 100 | 6.94 | 100 |
| I.V. | 50 | 2.48 | 86.7 | 81.8 | 117.9 |
| p.o. | 20 | 0.0426 | 3.72 | 3.70 | 13.3 |
| p.o. | 50 | 0.133 | 4.65 | 15.4 | 22.2 |
| p.o. | 100 | 0.526 | 9.20 | 73.8 | 53.2 |
| Gb | 1 | 0.00826 | 14.4 | | — |
| Gb | 3 | 0.0468 | 27.3 | 0.389 | 9.34 |
| Gb | 10 | 0.200 | 35.0 | 3.81 | 27.4 |
| Gb | 30 | 1.27 | 74.0 | 22.9 | 55 |
| Gb | 100 | 5.84 | 102 | 223 | 161 |

“Cmax” is the mean maximum plasma concentration; “F Cmax” is the calculated mean bioavailability of the Cmax for propofol generated from the tested prodrug (as administered via various routes and at various doses), relative to an intravenous dose of 5 mg/kg at Cmax; “AUCt” is the mean area under the curve from time 0 to the last measured time point (360 min); “F AUCt” is the calculated mean bioavailability for AUCt. “Bioavailability” is the quotient, expressed as per cent, of the Cmax or AUCt for intragastric (po) or intraduodenal (GB) administration, and the Cmax or AUCt for a 5 mg/kg intravenous dose, adjusted for dose. For example, for a 30 mg/kg gastric bypass administration of the tested prodrug, the propofol bioavailability F Cmax is calculated by dividing Cmax_{30gb} (1.27 μg/mL plasma) by the 5 mg/kg I.V. Cmax (0.286 μg/mL plasma)=4.44; and by dividing that quotient by 6 (since the gastric-bypass administered dose of the prodrug was 6-fold higher than the 5mg/kg I.V.-administered dose)=0.74. This transformation provides a reasonable measure to compare the systemic exposure to propofol generated from the test compound (after administration of various doses by various routes) to systemic propofol exposure after I.V. administration of the prodrug.

As is apparent from table I, the Cmax bioavailability of propofol derived from the intragastrally administered test compound (p.o.) is limited at all tested dose levels (see F Cmax for 20, 50, and 100 mg/kg p.o.). However, when the area under the curve is measured, oral bioavailability compares favorably to intravenous bioavailability, especially at the higher oral dose level, where it is above 50% of that of an intravenous dose (see F AUCt for 20, 50, and 100 mg/kg p.o.). This finding is consistent with the observation that oral doses of the prodrug delivered directly into the stomach cause a later, lower peak concentration of propofol in plasma (Cmax), but that appreciable plasma levels are sustained over longer periods of time compared to the other two routes of administration.

Consistent with the prodrug's high pharmacological efficacy when administered directly into the duodenum in the above-described behavioral pharmacology experiments, bioavailability of propofol liberated from the prodrug was found to be markedly enhanced when administered via gastric bypass. When calculated for Cmax and AUCt, the bioavailability of propofol liberated from the prodrug approaches that of, and at higher dose levels is essentially indistinguishable from, propofol generated after intravenous doses of the prodrug (see F Cmax and F AUCt for 10, 30, and 100 mg/kg gastric bypass [“gb”]).

These experiments demonstrate that the experimental compound is capable of causing a sedated/anesthetized state, the onset of which is about equally rapid with oral, intravenous, or intraduodenal administration, although peak effects may be delayed with oral/intragastric administration. The observed pharmacological effects are dose-dependent with all three routes of administration. Based on these experiments, it is concluded that the experimental compound is bioavaliable and biologically active when given by each route of administration. In the case of oral/intragastric administration, the biological potency of the peak effect in the described experimental paradigm is approximately 10% of that observed for intravenous administration, although the observed effects can be longer-lasting.

Notably, when the stomach was bypassed, considerably lower doses of the test compound were required to achieve the observed behavioral effects as compared to oral gavage. This finding is consistent with the pharmacokinetic profile of propofol released from the prodrug after gastric bypass administration (table I). Delivery of the test compound directly into the gut also allowed for doses approaching those required for intravenous delivery. In a comparison of FIGS. 1 and 2 with FIG. 4, for example, it is apparent that administration of 100 mg/kg intraduodenally, and 400 mg/kg intragastrally, were both sufficient to cause a substantially similar pharmacological effect. A comparison of FIG. 3 with FIG. 4, for example, reveals that both intravenous administration of 40 mg/kg, and intraduodenal administration of 50 mg/kg of the test compound are sufficient to cause a substantially similar pharmacological effect. By studying and extrapolating the dose-activity relationship of FIGS. 1-4, it can further be expected that intraduodenal administration of about 40 mg/kg would be sufficient to cause a pharmacological effect substantially similar to that caused by IV administration of 20-30 mg/kg. Thus, it is concluded from the above experiments that the experimental compound, O-phosphonooxymethyl propofol disodium salt, displays a favorable pharmacological profile as an orally ingestible agent, for example as a sedative/hypnotic drug.

### EXAMPLE 2

In a crossover study aimed at determining the plasma bioavailability of propofol after oral or intraduodenal administration of O-phosphonooxymethyl propofol disodium salt, seven male human volunteers received 400 mg of the test compound dissolved in aqueous solution at a concentration of 35 mg/ml via the oral route, and, on a separate occasion, via an endoscopic catheter directly into the duodenal lumen. Blood plasma samples were drawn at various time points post administration and frozen until chromatographic analysis of propofol concentrations. Plasma propofol concentrations at various time points after administration are given for both routes of administration in Table II, below:

TABLE II
Blood plasma concentrations of propofol in human volunteers at
various time points following oral or intraduodenal (ID) administration
of 400 mg O-phosphonooxymenthyl propofol disodium salt

| | | Time (hr) | | | | | | | | | | | |
|---|---|---|---|---|---|---|---|---|---|---|---|---|---|
| Route | | 0.0 | 0.08 | 0.17 | 0.33 | 0.50 | 0.75 | 1.0 | 1.5 | 2.0 | 4.0 | 6.0 | 9.0 |
| Oral | Median | 0.0 | 0.0 | 43.6 | 153.6 | 151.4 | 71.1 | 37.6 | 22.8 | 18.8 | 6.6 | 0.0 | 0.0 |
| | Mean | 0.0 | 6.6 | 103.9 | 197.2 | 144.1 | 75.0 | 53.2 | 32.3 | 19.0 | 6.2 | 0.9 | 0.9 |
| | SD | 0.0 | 9.4 | 117.8 | 126.1 | 47.9 | 17.8 | 42.2 | 21.3 | 8.4 | 2.9 | 2.4 | 2.3 |
| ID | Median | 0.0 | 144.5 | 210.9 | 277.3 | 211.3 | 52.3 | 45.3 | 24.7 | 14.2 | 5.6 | 0.0 | 0.0 |
| | Mean | 0.0 | 194.1 | 247.6 | 272.0 | 178.6 | 76.5 | 47.7 | 25.4 | 16.4 | 5.0 | 0.0 | 0.0 |
| | SD | 0.0 | 167.4 | 139.4 | 112.0 | 78.9 | 38.3 | 16.0 | 7.8 | 5.1 | 3.8 | 0.0 | 0.0 |

Some of the plasma propofol concentrations from four of the subjects were found to be above the upper limit of quantification (400 ng/ml) of the assay for the second, third, and fourth timepoints, and were assumed to be 400 ng/ml for purposes of calculating mean plasma concentrations in this analysis.

The results of this study show that oral administration of O-phosphonooxymethyl propofol disodium salt caused appreciable plasma concentrations of propofol in human subjects, making it a suitable therapeutic agent for the treatment of medical conditions which are amenable to treatment with propofol, but having the advantage of being administrable via the therapeutically convenient oral route. Moreover, when a similar amount of the prodrug was administered by intraduodenal instillation, plasma propofol released from the prodrug was detected at appreciably higher levels, and at earlier timepoints, as compared to oral administration. This finding confirms that therapeutically equivalent propofol plasma concentrations can be achieved by oral/intragastric and intraduodenal administration of the prodrug, and that the dose of the prodrug for intraduodenal administration can be reduced from the levels needed with oral/intragastric administration to achieve such therapeutically equivalent propofol plasma levels.

The invention being thus described and illustrated, it will be understood by those skilled in the art that the particular examples and embodiments can be modified in many ways without significantly departing from the scope and substance of this invention. The present application contemplates any and all such modifications.

We claim:

1. A method of treating or preventing migraine pain in a patient, comprising: orally administering to said patient a therapeutically sufficient amount of O-phosphonooxymethyl propofol disodium salt formulated in an enteric coated tablet or capsule, said tablet or capsule being specifically adapted to release said therapeutically sufficient amount directly into the gut after passage through the upper alimentary tract and stomach.

2. A method of administering a compound to a patient in need thereof, comprising: orally administering the compound in an amount sufficient to cause a pharmacological effect in said patient, wherein the compound is of formula I, and is capable of causing a substantially similar pharmacological effect when administered intravenously in a lower amount; wherein said orally administered amount is higher than the amount sufficient to cause the substantially similar pharmacological effect by intravenous administration of said compound; formula I being: 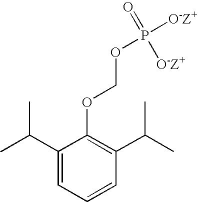
each Z being independently selected from the group consisting of hydrogen, alkali metal ion, and amine.

3. The method of claim 2, wherein said compound is formulated in a solid oral dosage form specifically adapted to release the compound into the stomach.

4. The method of claim 2, wherein said compound is formulated in a solid oral dosage form specifically adapted to release the compound into the gut after passage through the upper alimentary tract.

5. The method of claim 4, wherein said formulation is in the form of an enteric coated tablet, enteric coated capsule, a capsule comprising enteric coated granules or particles, or a tablet containing enteric coated granules or particles.

6. The method of claim 2, wherein said pharmacological effect is selected from the group consisting of: inducing or maintaining an unconscious state; inducing or maintaining a conscious sedated state; inducing or maintaining a somnolent state, treating insomnia, treating sleep disorders characterized by inappropriate wakefulness; treating anxiety; treating nausea or vomiting; treating itching associated with a pruritic condition; treating an epileptic condition; treating migraine pain; treating cluster headaches, treating refractory headaches, treating other acute headaches, treating trigeminal facial pain, treating dental pain, treating neuropathic pain, treating phantom limb pain; treating postoperative pain; treating inflammatory pain; treating neurogenic pain; treating arthritic pain; treating or preventing neurodegeneration caused by traumatic or vascular or toxic injury or by disease, treating spasticity; treating hyperekplexia; treating tetanus; and causing a muscle relaxant effect.

7. The method of claim 2, wherein said pharmacological effect is inducing or maintaining an unconscious state and said orally administered amount is from about 100 mg/kg to about 1,000 mg/kg.

8. The method of claim 7, wherein said orally administered amount is from about 200 mg/kg to about 600 mg/kg.

9. The method of claim 7, wherein said orally administered amount is from about 250 mg/kg to about 500 mg/kg.

10. The method of claim 2, wherein said pharmacological effect is inducing or maintaining a somnolent state and said orally administered amount is from about 10 mg/kg to about 300 mg/kg.

11. The method of claim 10, wherein said orally administered amount is from about 20 mg/kg to about 250 mg/kg.

12. The method of claim 10, wherein said orally administered amount is from about 25 mg/kg to about 200 mg/kg.

13. The method of claim 2, wherein said pharmacological effect is treating nausea or vomiting and said orally administered amount is from about 2 mg/kg to about 250 mg/kg.

14. The method of claim 13, wherein said orally administered amount is from about 5 mg/kg to about 200 mg/kg.

15. The method of claim 13, wherein said orally administered amount is from about 5 mg/kg to about 150 mg/kg.

16. The method of claim 13, wherein said orally administered amount is from about 7.5 mg/kg to about 30 mg/kg.

17. The method of claim 2, wherein said pharmacological effect is treating itching associated with a pruritic condition and said orally administered amount is from about 2 mg/kg to about 250 mg/kg.

18. The method of claim 17, wherein said orally administered amount is from about 5 mg/kg to about 200 mg/kg.

19. The method of claim 17, wherein said orally administered amount is from about 5 mg/kg to about 150 mg/kg.

20. The method of claim 17, wherein said orally administered amount is from about 7.5 mg/kg to about 30 mg/kg.

21. The method of claim 2, wherein said pharmacological effect is treating an epileptic condition and said orally administered amount is from about 2 mg/kg to about 400 mg/kg.

22. The method of claim 21, wherein said orally administered amount is from about 5 mg/kg to about 300 mg/kg.

23. The method of claim 21, wherein said orally administered amount is from about 5 mg/kg to about 200 mg/kg.

24. The method of claim 21, wherein said orally administered amount is from about 7.5 mg/kg to about 60 mg/kg.

25. The method of claim 2, wherein said pharmacological effect is treating migraine pain, cluster headaches, refractory headaches and other acute headaches and said orally administered amount is from about 2 mg/kg to about 300 mg/kg.

26. The method of claim 25, wherein said orally administered amount is from about 5 mg/kg to about 250 mg/kg.

27. The method of claim 25, wherein said orally administered amount is from about 5 mg/kg to about 200 mg/kg.

28. The method of claim 25, wherein said orally administered amount is from about 10 mg/kg to about 30 mg/kg.

29. The method of claim 2, wherein said pharmacological effect is treating spasticity, hyperekplexia, tetanus and causing a muscle relaxant effect and said orally administered amount is from about 10 mg/kg to about 350 mg/kg.

30. The method of claim 29, wherein said orally administered amount is from about 30 mg/kg to about 200 mg/kg.

31. The method of claim 29, wherein said orally administered amount is from about 40 mg/kg to about 80 mg/kg.

32. A method of administering a compound to a patient in need thereof, which comprises: introducing the compound directly into the gut in an amount sufficient to cause a pharmacological effect in said patient; wherein the compound is of Formula I: 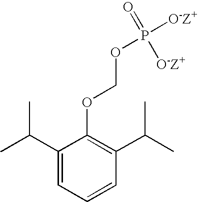
each Z being independently selected from the group consisting of hydrogen, alkali metal ion, and amine.

33. The method of claim 32, wherein said compound is administered orally, and is formulated in a solid oral dosage form specifically adapted to release the compound into the gut after passage through the upper alimentary tract.

34. The method of claim 33, wherein said formulation is in the form of an enteric coated tablet, enteric coated capsule, a capsule comprising enteric coated granules or particles, or a tablet containing enteric coated granules or particles.

35. The method of claim 32, wherein said pharmacological effect is selected from the group consisting of: inducing or maintaining an unconscious state; inducing or maintaining a conscious sedated state; inducing or maintaining a somnolent state, treating insomnia, treating sleep disorders characterized by inappropriate wakefulness; treating anxiety; treating nausea or vomiting; treating itching associated with a pruritic condition; treating an epileptic condition; treating migraine pain; treating cluster headaches, treating refractory headaches, treating other acute headaches, treating trigeminal facial pain, treating dental pain, treating neuropathic pain, treating phantom limb pain; treating postoperative pain; treating inflammatory pain; treating neurogenic pain; treating arthritic pain; treating or preventing neurodegeneration caused by traumatic or vascular or toxic injury or by disease; treating spasticity; treating hyperekplexia; treating tetanus; and causing a muscle relaxant effect.

36. The method of claim 32, wherein said pharmacological effect is inducing or maintaining an unconscious state and said administered amount is from about 15 mg/kg to about 500 mg/kg.

37. The method of claim 36, wherein said amount is from about 20 mg/kg to about 400 mg/kg.

38. The method of claim 36, wherein said amount is from about 25 mg/kg to about 300 mg/kg.

39. The method of claim 32, wherein said pharmacological effect is inducing or maintaining a somnolent state and said administered amount is from about 1 mg/kg to about 75 mg/kg.

40. The method of claim 39, wherein said amount is from about 2 mg/kg to about 50 mg/kg.

41. The method of claim 39, wherein said amount is from about 5 mg/kg to about 40 mg/kg.

42. The method of claim 32, wherein said pharmacological effect is treating nausea or vomiting and said administered amount is from about 1 mg/kg to about 50 mg/kg.

43. The method of claim 42, wherein said amount is from about 2 mg/kg to about 30 mg/kg.

44. The method of claim 42, wherein said amount is from about 2 mg/kg to about 20 mg/kg.

45. The method of claim 42, wherein said amount is from about 3.5 mg/kg to about 12.5 mg/kg.

46. The method of claim 32, wherein said pharmacological effect is treating itching associated with a pruritic condition and said administered amount is from about 1 mg/kg to about 50 mg/kg.

47. The method of claim 46, wherein said amount is from about 2 mg/kg to about 30 mg/kg.

48. The method of claim 46, wherein said amount is from about 2 mg/kg to about 20 mg/kg.

49. The method of claim 46, wherein said amount is from about 3.5 mg/kg to about 12.5 mg/kg.

50. The method of claim 32, wherein said pharmacological effect is treating an epileptic condition and said administered amount is from about 1 mg/kg to about 100 mg/kg.

51. The method of claim 50, wherein said amount is from about 1 mg/kg to about 75 mg/kg.

52. The method of claim 50, wherein said amount is from about 2 mg/kg to about 50 mg/kg.

53. The method of claim 50, wherein said amount is from about 3.5 mg/kg to about 25 mg/kg.

54. The method of claim 32, wherein said pharmacological effect is treating migraine pain, cluster headaches, refractory headaches and other acute headaches and said administered amount is from about 1 mg/kg to about 75 mg/kg.

55. The method of claim 54, wherein said amount is from about 1 mg/kg to about 50 mg/kg.

56. The method of claim 54, wherein said amount is from about 2 mg/kg to about 30 mg/kg.

57. The method of claim 54, wherein said amount is from about 5 mg/kg to about 20 mg/kg.

58. The method of claim 32, wherein said pharmacological effect is treating spasticity, hyperekplexia, tetanus and causing a muscle relaxant effect and said administered amount is from about 5 mg/kg to about 200 mg/kg.

59. The method of claim 58, wherein said amount is from about 20 mg/kg to about 125 mg/kg.

60. The method of claim 58, wherein said amount is from about 30 mg/kg to about 50 mg/kg.
